(19) United States
(12) Patent Application Publication
(10) Pub. No.: US 2009/0163814 A1
(43) Pub. Date: Jun. 25, 2009

(54) ULTRASONIC DIAGNOSTIC APPARATUS AND OPERATING DEVICE THEREOF

(75) Inventors: Shinichi AMEMIYA, Tokyo (JP); Masashi SEKI, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(73) Assignee: GE MEDICAL SYSTEMS GLOBAL TECHNOLOGY COMPANY, LLC, Waukesha, WI (US)

(21) Appl. No.: 12/338,737

(22) Filed: Dec. 18, 2008

(30) Foreign Application Priority Data
Dec. 21, 2007 (JP) 2007-330247

(51) Int. Cl. A61B 8/00

(52) U.S. Cl. 600/443

(57) ABSTRACT

An ultrasonic diagnostic apparatus includes an operating unit for enabling the operator to input instructions, a signal processing unit which produces ultrasonic image data on the basis of echo signals obtained by transmitting an ultrasonic wave, and an image display unit on which an ultrasonic image based on the ultrasonic image data produced by the signal processing unit is displayed. Said operating unit is configured as a device separate at least from said image display unit, and said operating device is enabled to display an ultrasonic image.

### CROSS REFERENCE TO RELATED APPLICATIONS

This application claims the benefit of Japanese Patent Application No. 2007-330247 filed Dec. 21, 2007, which is hereby incorporated by reference in its entirety.

### BACKGROUND OF THE INVENTION

The embodiments described herein relate to an ultrasonic diagnostic apparatus that produces an ultrasonic image on the basis of echo signals obtained by transmitting an ultrasonic wave, and an operating device thereof.

Conventionally known ultrasonic diagnostic apparatuses include an ultrasonic diagnostic apparatus provided with an operating unit for enabling the operator to input instructions, a signal processing unit which produces ultrasonic image data on the basis of echo signals obtained by transmitting an ultrasonic wave in accordance with an instruction inputted with this operating unit, and an image display unit on which an ultrasonic image based on the ultrasonic image data is displayed, and these operating unit, signal processing unit and image display unit are integrally configured (see for instance Japanese Unexamined Patent Publication No. 2003-339708).

The ultrasonic diagnostic apparatus is usually installed beside a subject when it is used, such as on the bedside. Then, the operator positioned near the ultrasonic diagnostic apparatus on the bedside performs scanning with a probe kept in contact with the subject lying on the bed while watching the ultrasonic image displayed on the image display unit, and accordingly is obliged to perform scanning in an unnatural bodily posture. Accordingly, in order to prevent the operator from being taking such an unnatural bodily posture when he or she performs scanning, ultrasonic diagnostic apparatuses in which the image display unit is separated from the operating unit to make them separate devices to enable the image display unit to be in front of the operator facing the subject to perform scanning are proposed (see for instance Japanese Unexamined Patent Publication No. 2002-85405).

Incidentally, it is a conventional practice to display a cursor over the ultrasonic image displayed on the image display unit and to measure the size of a tumor or the like on the ultrasonic image by using this cursor. In this case, the operator moves the cursor to designate the measuring range by operating the operating device placed on the bedside while watching the ultrasonic image displayed on the image display unit. Then, if the operating device is placed on the bedside as described above and the image display unit is arranged in front of the operator facing the subject lying on the bed, the operator will operate the operating unit with his or her body in a twisted state, obliged to work in an unnatural bodily posture.

It is desirable that the problem described previously is solved.

### BRIEF DESCRIPTION OF THE INVENTION

The invention according to a first aspect provides an ultrasonic diagnostic apparatus including: an operating unit for enabling the operator to input instructions; a signal processing unit which produces ultrasonic image data on the basis of echo signals obtained by transmitting an ultrasonic wave; and an image display unit on which an ultrasonic image based on the ultrasonic image data produced by the signal processing unit is displayed, the ultrasonic diagnostic apparatus being characterized in that the operating unit is configured as a device separate at least from the image display unit, and the operating device is enabled to display an ultrasonic image.

The invention according to a second aspect provides the ultrasonic diagnostic apparatus according to the first aspect, including: a data transmitting unit which transmits ultrasonic image data produced by the signal processing unit to the operating unit, wherein the operating device further includes: a data receiving unit which receives the ultrasonic image data from the data transmitting unit; and a display unit on which an ultrasonic image based on the ultrasonic image data received by the data receiving unit is displayed.

The invention according to a third aspect provides the ultrasonic diagnostic apparatus according to the second aspect characterized in that the display unit displays buttons for enabling the operator to input instructions.

The invention according to a fourth aspect provides the ultrasonic diagnostic apparatus according to the second or third aspect characterized in that the data transmitting unit and the data receiving unit perform radio communication.

The invention according to a fifth aspect provides the ultrasonic diagnostic apparatus according to any of the second through fourth aspects characterized in that the operating device has a buffer memory for temporarily storing ultrasonic image data received by the data receiving unit, and an ultrasonic image based on the ultrasonic image data stored in the buffer memory is displayed on the display unit.

The invention according to a sixth aspect provides the ultrasonic diagnostic apparatus according to any of the second through fifth aspects including: a display setting unit for displaying on an ultrasonic image a measurement range designating display which designates the measurement range of the object of measurement in the ultrasonic image; and a computing unit which computes measured values on the basis of the measurement range designating display, wherein the operating device has input means for moving the measurement range designating display and designating the measurement range.

The invention according to a seventh aspect provides the ultrasonic diagnostic apparatus according to the second aspect including: a processing device having the signal processing unit and the data transmitting unit, wherein the processing device is a separate body from the operating device.

The invention according to an eighth aspect provides the ultrasonic diagnostic apparatus according to the sixth aspect including: a processing device having the signal processing unit, the data transmitting unit, the display setting unit and the computing unit, wherein the processing device is a separate body from the operating device.

The invention according to a ninth aspect provides the ultrasonic diagnostic apparatus according to any of the first through eighth aspects characterized in that: the operating unit is equipped with a loudspeaker; Doppler sound data produced by the signal processing unit by taking out Doppler components from the echo signals are transmitted to the operating unit; and Doppler sounds based on these Doppler sound data are reproduced by the loudspeaker.

The invention according to a tenth aspect provides the ultrasonic diagnostic apparatus according to any of the first through ninth aspects characterized in that the ultrasonic image displayed on the operating device is a moving image.

The invention according to an eleventh aspect provides an operating device of an ultrasonic diagnostic apparatus, the operating device being intended to enable the operator to input instructions, constituting part of the ultrasonic diagnostic apparatus including: a signal processing unit which produces ultrasonic image data on the basis of echo signals obtained by transmitting an ultrasonic wave; and an image display unit on which an ultrasonic image based on the ultrasonic image data produced by the signal processing unit is displayed, and being a body separate at least from the image display unit, characterized in that it is enabled to display an ultrasonic image.

The invention according to a twelfth aspect provides the operating device of an ultrasonic diagnostic apparatus according to the eleventh aspect including: a data receiving unit which receives the ultrasonic image data produced by the signal processing unit; and a display unit on which an ultrasonic image based on the ultrasonic image data received by the data receiving unit is displayed.

The invention according to a thirteenth aspect provides the operating device of an ultrasonic diagnostic apparatus according to the twelfth aspect further including a buffer memory for temporarily storing ultrasonic image data received by the data receiving unit, wherein an ultrasonic image based on the ultrasonic image data stored in the buffer memory is displayed on the display unit.

According to the invention in the first aspect, as an ultrasonic image is displayed on the operating device, even if the operating device is a separate body from the image display unit, the operator can operate the operating device while watching the ultrasonic image displayed on the operating device, and accordingly can work in a natural bodily posture.

According to the invention in the second aspect, as an ultrasonic image based on the ultrasonic image data transmitted from the data transmitting unit and received by the data receiving unit of the operating device is displayed on the display unit of the operating device, the operator can operate the operating device in a natural bodily posture while watching this ultrasonic image.

According to the invention in the third aspect, as an ultrasonic image is displayed on the display unit displaying buttons for enabling the operator to input instructions, the operator can operate the operating device in a natural bodily posture while watching this ultrasonic image.

According to the invention in the fourth aspect, as an ultrasonic image based on the ultrasonic image data transmitted from the data transmitting unit by radio communication and received by the data receiving unit of the operating device is displayed on the display unit of the operating device, the operator can operate the operating device in a natural bodily posture while watching this ultrasonic image.

According to the invention in the fifth aspect, an ultrasonic image based on the ultrasonic image data temporarily stored in the buffer memory is displayed on the display unit of the operating device, and the operator can operate the operating device in a natural bodily posture while watching this ultrasonic image.

According to the invention in the sixth aspect, as the measurement range is designated with a measurement range designating display on the ultrasonic image and, when the measured values are to be computed with the computing unit, the operator designates the measurement range by manipulating the input means of the operating device by moving the measurement range designating display while watching the ultrasonic image displayed on the operating device, the operator can work in a natural bodily posture.

According to the invention in the seventh aspect, as an ultrasonic image based on the ultrasonic image data produced by the signal processing unit and transmitted from the processing device is displayed on the operating device, the operator can operate the operating device while watching this ultrasonic image displayed on the operating device, and accordingly the operator can work in a natural bodily posture.

According to the invention in the eighth aspect, as an ultrasonic image based on the ultrasonic image data produced by the signal processing unit and transmitted from the processing device to the operating device is displayed on the operating device, the operator can operate the operating device while watching this ultrasonic image displayed on the operating device, and accordingly the operator can work in a natural bodily posture. Further, it is possible to have the display setting unit of the processing device display the measurement range designating display on the ultrasonic image and have the computing unit compute the measured values on the basis of this measurement range designating display.

According to the invention in the ninth aspect, it is possible to reproduce Doppler sounds from the loudspeaker of the operating device.

According to the invention in the tenth aspect, it is possible to display an ultrasonic moving image on the operating device.

According to the invention in the eleventh aspect, as an ultrasonic image is displayed on the operating device, even if the operating device is a separate body from the image display unit, the operator can operate the operating device while watching the ultrasonic image displayed on the operating device, and accordingly can work in a natural bodily posture.

According to the invention in the twelfth aspect, as an ultrasonic image based on the ultrasonic image data received by the data receiving unit is displayed on the display unit of the operating device, the operator can operate the operating device in a natural bodily posture while watching this ultrasonic image.

According to the invention in the thirteenth aspect, an ultrasonic image based on the ultrasonic image data temporarily stored in the buffer memory is displayed on the display unit, and the operator can operate the operating device in a natural bodily posture while watching this ultrasonic image.

Further objects and advantages of the present invention will be apparent from the following description of embodiments of the invention as illustrated in the accompanying drawings.

### BRIEF DESCRIPTION OF THE DRAWINGS

FIG. 1 is a partially exploded view showing an ultrasonic diagnostic apparatus.

FIG. 2 is a front view of the ultrasonic diagnostic apparatus shown in FIG. 1.

FIG. 3 is a plan of the ultrasonic diagnostic apparatus shown in FIG. 1.

FIG. 4 is a view showing the inside of the cable accommodating space.

FIG. 5 is a block diagram showing one example of configuration of the ultrasonic diagnostic apparatus shown in FIG. 1.

FIG. 6 is a block diagram showing one example of configuration of the transmission/reception unit.

FIG. 7 is a block diagram showing the configuration of the B-mode processing unit.

FIG. 8 is a block diagram showing one example of configuration of the Doppler processing unit.

FIG. 9 is a block diagram showing one example of configuration of the image processing unit.

FIG. 10 is a block diagram showing one example of configuration of the measurement information display processing unit.

FIG. 11 is a diagram showing a pair of cursors and the line segment connecting these cursors displayed on an ultrasonic image.

### DETAILED DESCRIPTION OF THE INVENTION

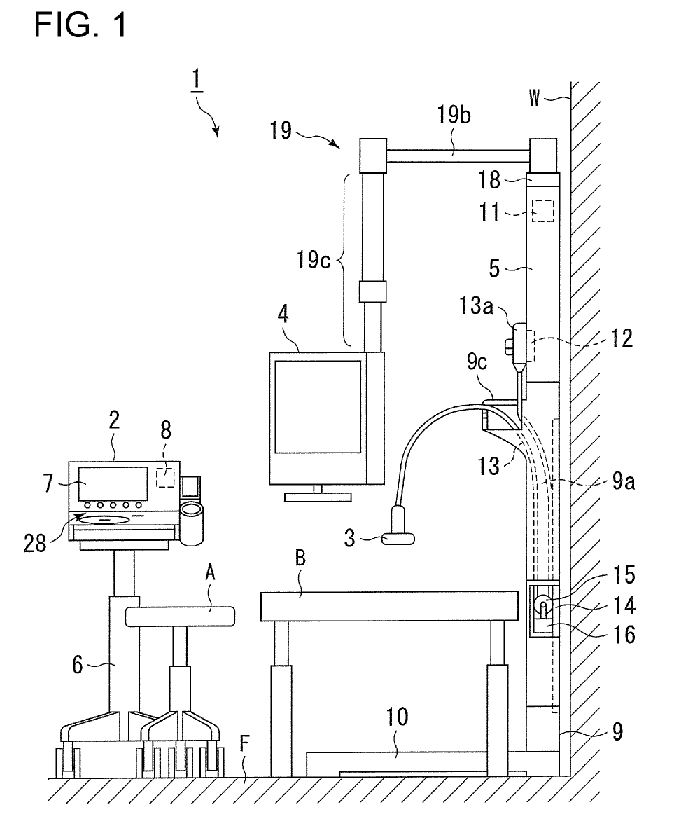

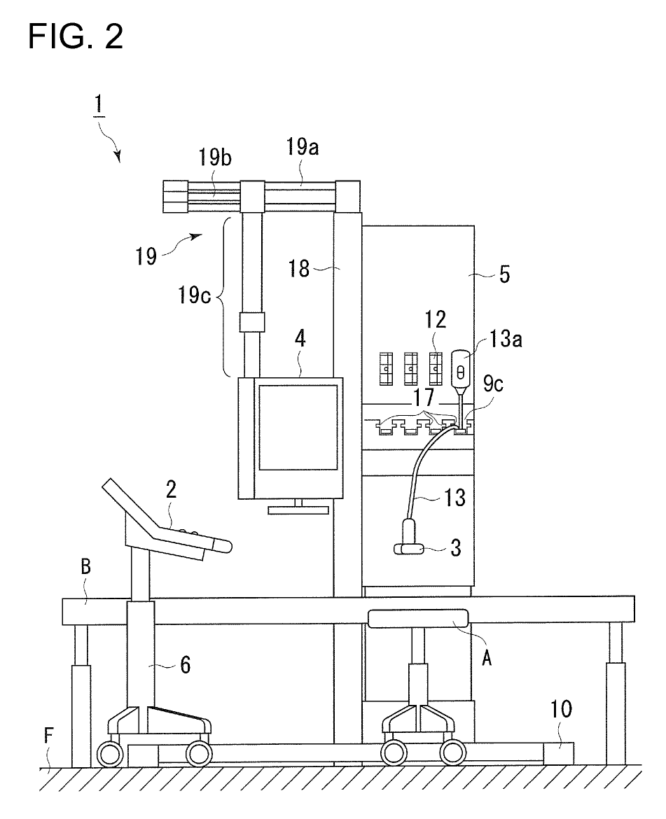

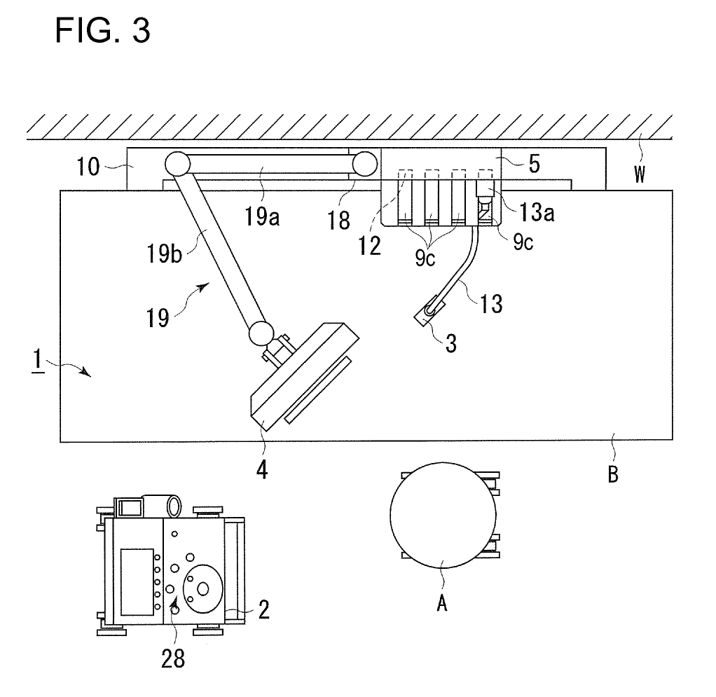

The invention will be described in further detail below with reference to embodiments illustrated in drawings. FIG. 1 is a partially exploded view showing an ultrasonic diagnostic apparatus pertaining to a mode for implementation of the invention; FIG. 2, a front view of the ultrasonic diagnostic apparatus shown in FIG. 1; and FIG. 3, a plan of the ultrasonic diagnostic apparatus shown in FIG. 1.

An ultrasonic diagnostic apparatus 1 is provided with an operating device 2 for enabling the operator to input instructions, a probe 3 for transmitting and receiving ultrasonic waves, an image display device 4 for displaying an ultrasonic image, and a processing device 5 for performing control to produce an ultrasonic image on the basis of echo signals obtained by driving the probe 3 in accordance with an instruction by the operator and to display it on the image display device 4.

The operating device 2, which is a separate body from the image display device 4 and the processing device 5, is installed on a caster-equipped movable table 6, and placed on the side where a chair A on which the operator is sit relative to a bed B on which the subject is laid.

The operating device 2 also has a display unit 7 which displays touch panel type operating buttons (not shown) for enabling the operator to input instructions. The ultrasonic image produced by the processing device 5 is also displayed on this display unit 7 besides the operating buttons. Further, the operating device 2 has operating device side radio communication unit 8 for radio communication with the processing device 5. This operating device side radio communication unit 8 is intended for receiving ultrasonic image data from the processing device side radio communication unit 11 (to be described afterwards) of the processing device 5, and constitutes an example of mode for implementing a data receiving unit according to the invention.

The processing device 5 is fixed onto a mount 9 placed between the bed B and the wall W. This mount 9 has a tip-resistant leg 10. It is desirable for this tip-resistant leg 10 to be fixed to a floor face F with anchor bolts (not shown).

The processing device 5 further has a processing device side radio communication unit 11 for use in radio communication with the operating device 2. This processing device side radio communication unit 11, intended for transmitting ultrasonic image data to the operating device 2, is one example of mode for implementing a data transmitting unit according to the invention. The processing device 5 further has a probe connector 12 for connecting a cable connector 13a of the probe 3.

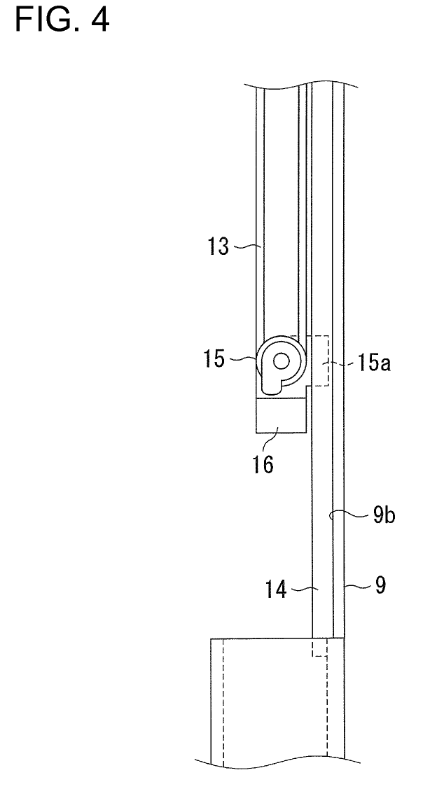

The inside of the mount 9 constitutes a cable accommodating space 9a for accommodating a cable 13 of the probe 3. FIG. 4 shows the inside of the cable accommodating space 9a. Within this cable accommodating space 9a, a rail 14 is disposed in a direction perpendicular to an inner wall face 9b. A running block 15 is held by the rail 14 via a holder 15a to be movable in the up-and-down directions. The running block 15 is pressured downward by a weight 16.

The cable 13, threaded round the running block 15 from above, is so accommodated in the cable accommodating space 9a as to form a U shape. The probe 3 side part of the cable 13 is pulled out of upper opening 9c of the mount 9. Incidentally, the cable 13 pulled out of the opening 9c can be held in an elastically deformed state in a slit 17 formed in the opening 9c.

The probe 3 has an array of a plurality of ultrasonic transducers not shown. Each individual ultrasonic transducer is configured of, for instance, a piezoelectric material such as PZT (lead zirconate titanate) ceramics.

The image display device 4, which is one example of mode for implementing of an image display unit according to the invention, is supported by the processing device 5 and an arm 19 extending from a strut 18 fixed to a side of the mount 9. This arm 19 includes a first horizontal arm 19a extending horizontally from the strut 18, a second horizontal arm 19b extending horizontally from this first horizontal arm 19a, and a perpendicular arm 19c extending perpendicularly from this second horizontal arm 19b. This perpendicular arm 19c is variable in length and can turn in the horizontal direction at its connecting part with the second horizontal arm 19b. The lower end of the perpendicular arm 19c constitutes a connecting part that can turn the image display device 4 within a perpendicular plane.

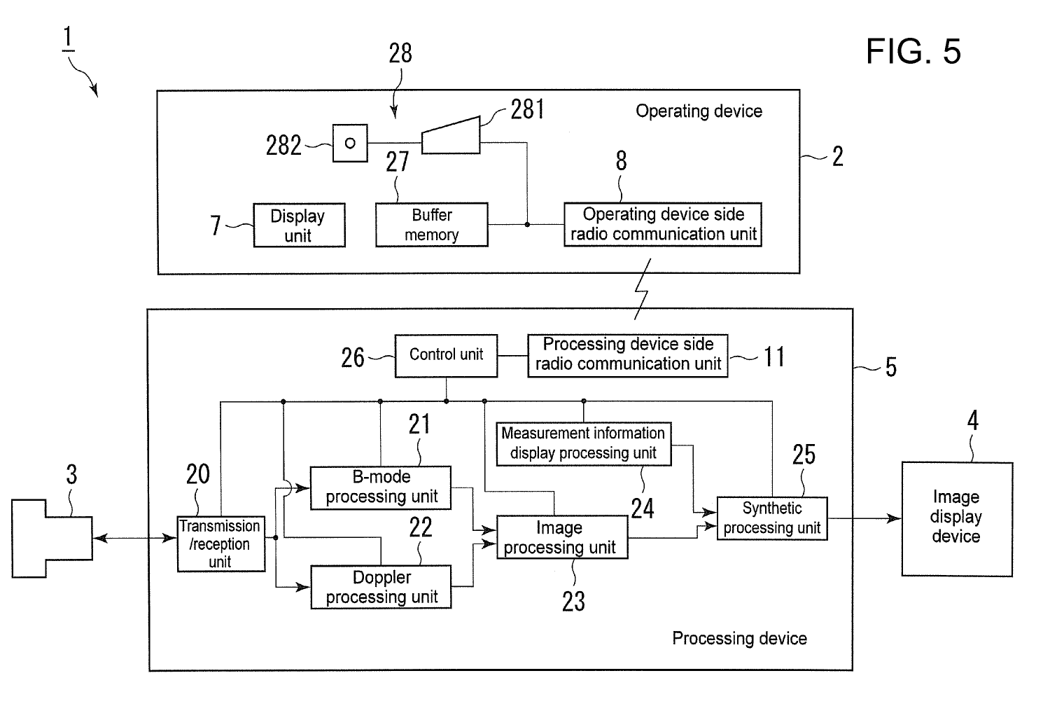

FIG. 5 is a block diagram showing one example of configuration of the ultrasonic diagnostic apparatus 1. The configuration of each part of the ultrasonic diagnostic apparatus 1 will be described in further detail with reference to this FIG. 5.

The processing device 5 has a transmission/reception unit 20, a B-mode processing unit 21, a Doppler processing unit 22, an image processing unit 23, a measurement information display processing unit 24, a synthetic processing unit 25 and a control unit 26 in addition to the processing device side radio communication unit 11. The operating device 2 has a buffer memory 27 and an input unit 28 in addition to the display unit 7 and the operating device side radio communication unit 8.

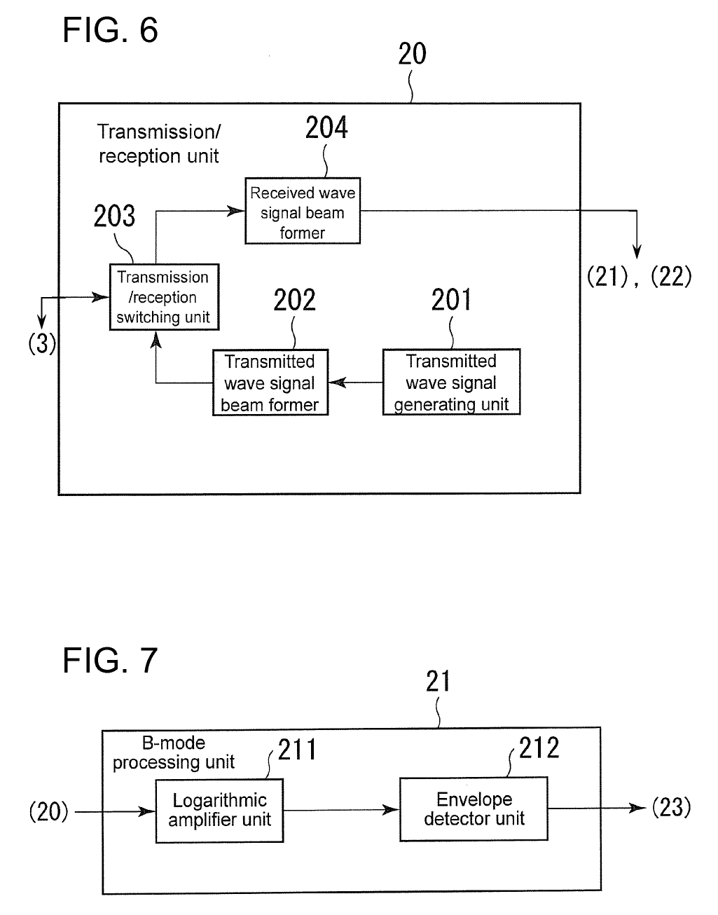

First, the transmission/reception unit 20 will be described. The probe 3 is connected to this transmission/reception unit 20. FIG. 6 is a block diagram showing one example of configuration of the transmission/reception unit 20. The transmission/reception unit 20 shown in this FIG. 6 has a transmitted wave signal generating unit 201, a transmitted wave signal beam former 202, a transmission/reception switching unit 203 and a received wave signal beam former 204.

The transmitted wave signal generating unit 201 periodically generates transmitted wave signals and inputs them to the transmitted wave signal beam former 202. The period of the transmitted wave signals is controlled by the control unit 26.

The transmitted wave signal beam former 202, which performs beam forming of the transmitted wave, generates a beam forming signal for forming an ultrasonic beam in a prescribed direction on the basis of transmitted wave signals. The beam forming signal consist of a plurality of drive signals assigned time differences corresponding to the direction. The beam forming is controlled by the control unit 26. The transmitted wave signal beam former 202 outputs the transmitted wave beam forming signal to the transmission/reception switching unit 203.

The transmission/reception switching unit 203 outputs the transmitted wave beam forming signal to the array of ultrasonic transducers. In this array of ultrasonic transducers, a plurality of ultrasonic transducers constituting a transmitted wave aperture generate ultrasonic waves each having a phase difference corresponding to the time difference of the drive signal. An ultrasonic beam along a sound ray in a prescribed direction is formed by the wave field synthesis of those ultrasonic waves.

The received wave signal beam former 204 is connected to the transmission/reception switching unit 203. The transmission/reception switching unit 203 outputs a plurality of echo signals received by a received wave aperture in the array of ultrasonic transducers to the received wave signal beam former 204.

The received wave signal beam former 204, which performs beam forming of the received wave matching the sound ray of the transmitted wave, regulates the phases of a plurality of received wave echoes by assigning time differences, and then adds them to generate echo signals along a sound ray in a prescribed direction. The beam forming of the received wave is controlled by the control unit 26.

The transmission/reception unit 20 is connected to the B-mode processing unit 21 and the Doppler processing unit 22. Echo signals for each sound ray outputted from the transmission/reception unit 20 are inputted to the B-mode processing unit 2 land the Doppler processing unit 22.

The B-mode processing unit 21 generates B-mode image data for each sound ray on the basis of echo signals. FIG. 7 is a block diagram showing one example of schematic configuration of the B-mode processing unit 21. The B-mode processing unit 21 shown in FIG. 7 has a logarithmic amplifier unit 211 and an envelope detector unit 212.

The B-mode processing unit 21 logarithmically amplifies echo signals with the logarithmic amplifier unit 211, performs envelope detection with the envelope detector unit 212 to obtain signals each representing the strength of the echo at each individual reflection point on a sound ray, namely an A scope signal, and generates B-mode image data with the amplitude of this A scope signal at each instant as the brightness of each.

The Doppler processing unit 22 is intended to generate Doppler image data for each sound ray on the basis of echo signals. The Doppler image data include flow velocity data, variance data and power data to be described afterwards.

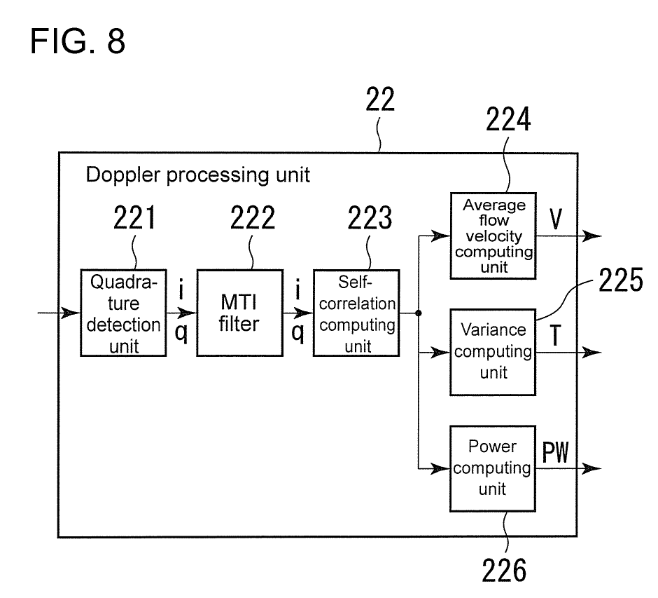

FIG. 8 is a block diagram showing one example of configuration of the Doppler processing unit 22. As shown in FIG. 8, the Doppler processing unit 22 has an quadrature detection unit 221, an MTI filter (moving target indication filter) 222, a self-correlation computing unit 223, an average flow velocity computing unit 224, a variance computing unit 225 and a power computing unit 226.

The Doppler processing unit 22 performs quadrature detection of echo signals with the quadrature detection unit 221 and subjects them to MTI processing with the MTI filter 222 to figure out the Doppler shift of the echo signals. It performs self-correlation computation of the output signals of the MTI filter 222 with the self-correlation computing unit 223. Then it figures out an average flow velocity from the result of self-correlation computation with the average flow velocity computing unit 224, figures out a variance T of the flow velocity V from the result of self-correlation computation with the variance computing unit 225, and figures out the power PW of Doppler signals from the result of self-correlation computation with the power computing unit 226. Hereinafter the average flow velocity may be referred to as simply the flow velocity. Also, the variance of the flow velocity may be referred to as simply the variance, and the power of Doppler signals, as simply the power.

Sets of data respectively representing the flow velocity V, the variance T and the power PW of the echo source moving within the subject are obtained for each sound ray by the Doppler processing unit 22. These sets of data respectively represent the flow velocity, the variance and the power of pixels on sound rays. The flow velocity is obtained as a component in the sound ray direction. Directions toward and away from the probe 3 are distinguished from each other.

The B-mode processing unit 21 and the Doppler processing unit 22 are connected to the image processing unit 23. This image processing unit 23 generates data of a B-mode image and of a Doppler image on the basis of data respectively inputted from the B-mode processing unit 21 and the Doppler processing unit 22, and further generates data of an image resulting from the synthesis of the B-mode image and the Doppler image. Each of the B-mode image, the Doppler image and the image resulting from their synthesis here is referred to as an ultrasonic image. The B-mode processing unit 21, the Doppler processing unit 22 and the image processing unit 23 constitute one example of mode of implementing the signal processing unit according to the invention.

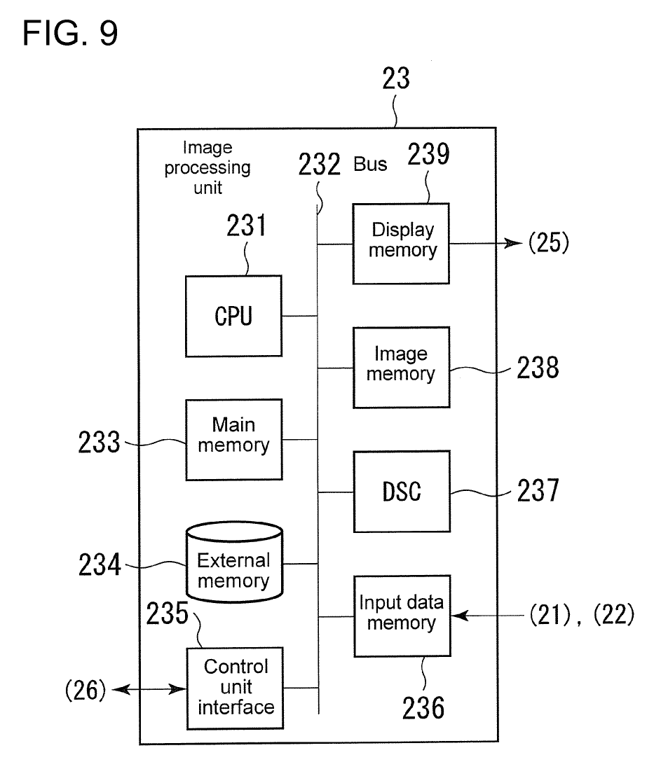

The image processing unit 23 will be described with reference to FIG. 9. FIG. 9 is a block diagram showing one example of configuration of the image processing unit 23. As shown in this FIG. 9, the image processing unit 23 has a central processing unit (CPU) 231. To this CPU 231, a main memory 233, an external memory 234, a control unit interface 235, an input data memory 236, a digital scan converter (DSC) 237, an image memory 238 and a display memory 239 are connected via a bus 232.

Programs to be executed by the CPU 231 are stored in the external memory 234. Various data to be used when the CPU 231 executes a program are also stored in the external memory 234.

The CPU 231 executes prescribed image processing by loading a program from the external memory 234 into the main memory 233 and executing it. In the process of program execution, the CPU 231 delivers and receives control signals to and from the control unit 26 via the control unit interface 235.

The B-mode image data and the Doppler image data inputted from the B-mode processing unit 21 and the Doppler processing unit 22 for each sound line are stored into the input data memory 236. Data in the input data memory 236 are scanned and converted by the DSC 237 and stored into the image memory 238. Data in the image memory 238 are outputted to the synthetic processing unit 25 via the display memory 239. The data then outputted from the image memory 238 are data of the ultrasonic image resulting from the synthesis of the B-mode image and the Doppler image.

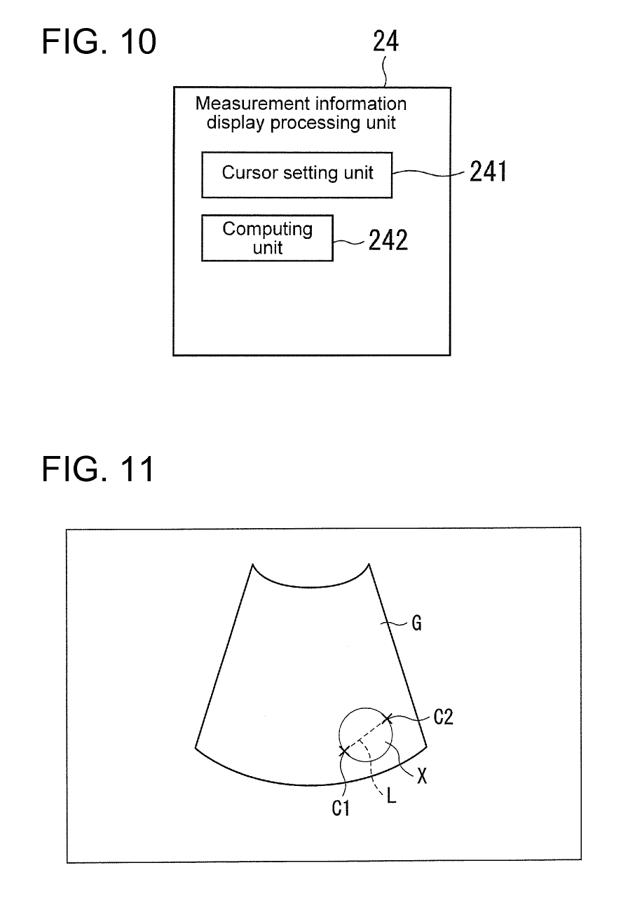

The measurement information display processing unit 24 has a cursor setting unit 241 and a computing unit 242 as shown in FIG. 10. The cursor setting unit 241 generates display data of a pair of cursors and a line segment L connecting these cursors for measuring the size of the object of measurement, for instance a tumor, on an ultrasonic image. FIG. 11 shows a pair of cursors C1 and C2 and the line segment L connecting these cursors C1 and C2 displayed on an ultrasonic image G. These cursors C1 and C2 designated the range of measuring the object of measurement on the ultrasonic image G, representing one example of mode for implementing the measurement range designation display according to the invention. The cursor setting unit 241 is one example of mode for implementing the display setting unit according to the invention. Incidentally, a tumor X is shown in FIG. 11 as the object of measurement.

The computing unit 242 computes the measured values in the measurement range designated by these cursors C1 and C2 on the basis of positional information on the cursors C1 and C2 on the ultrasonic image G displayed on the display unit 7. The positional information on the cursors C1 and C2 on the display unit 7 is transmitted from the operating device side radio communication unit 8 to the processing device side radio communication unit 11. Then, this positional information received by the processing device side radio communication unit 11 is inputted to the measurement information display processing unit 24 via the control unit 26.

The synthetic processing unit 25 is connected to the image processing unit 23 and the measurement information display processing unit 24. Data in the display memory 239 of the image processing unit 23 and display data on the cursors C1 and C2 and the line segment L from the measurement information display processing unit 24 are inputted to this synthetic processing unit 25. Then, the ultrasonic image is synthesized with the cursors C1 and C2 and the line segment L by the synthetic processing unit 25.

Data on the ultrasonic image synthesized with the cursors C1 and C2 and the line segment L by the synthetic processing unit 25 are outputted to the image display device 4, and also transmitted from the processing device side radio communication unit 11 to the operating device side radio communication unit 8 via the control unit 26.

On the image display device 4, to which data from the synthetic processing unit 25 are inputted via a cable (not shown) arranged in the arm 19, an ultrasonic image based on these data is displayed.

In the operating device 2, data on the ultrasonic image outputted from the synthetic processing unit 25 and transmitted from the processing device side radio communication unit 11 are received by the operating device side radio communication unit 8, and temporarily stored into the buffer memory 27. Then, an ultrasonic image based on the data stored in the buffer memory 27 is displayed on the display unit 7.

The input unit 28 of the operating device 2 has a keyboard 281 and a pointing device 282 such as a mouse or a track ball (no detailed configuration shown in FIGS. 1 through 3). The cursors C1 and C2 shown on the display unit 7 can be moved on the screen by operating the pointing device 282, and are positioned by pressing buttons on the keyboard 281. The input unit 28 is one example of input means in a mode for implementing the invention. When the cursors C1 and C2 are moved with the pointing device 282, positional information on them is transmitted from the operating device side radio communication unit 8 toward the processing device 5 and inputted to the cursor setting unit 241 via the processing device side radio communication unit 11 and the control unit 26.

Now will be described the operation of the ultrasonic diagnostic apparatus 1. First, the probe 3 is brought into contact with the subject, and the input unit 28 of the operating device 2 is manipulated to perform an image obtaining action by, for instance, combined use of the B-mode and the Doppler mode. This enables images in the B-mode and images in the Doppler mode to be obtained on a time sharing basis under the control of the control unit 26. Thus, for instance, mixed scanning in the B-mode and in the Doppler mode is accomplished at a rate of performing scanning in the B-mode every time scanning in the Doppler mode is performed a prescribed number of times.

In the B-mode, the transmission/reception unit 20 scans the inside of the subject in the sequence of sound rays via the probe 3, and successively receives the resultant echoes. The B-mode processing unit 21 generates B-mode image data for each sound ray on the basis of echo signals inputted from the transmission/reception unit 20.

The image processing unit 23 stores into the input data memory 236 the B-mode image data for each sound ray inputted from the B-mode processing unit 21. This results in the formation of a sound ray data space regarding the B-mode image data within the input data memory 236.

In the Doppler mode, the transmission/reception unit 20 scans the inside of the subject in the sequence of sound rays via the probe 3, and successively receives the resultant echoes. In that process, transmission of an ultrasonic wave and reception of an echo are done a plurality of times per sound ray.

The Doppler processing unit 22 figures out the flow velocity V, the variance T and the power PW on the basis of the echo signals. These calculated values serve as data representing the flow velocity, the variance and the power of the echo source for each sound ray and each pixel.

The image processing unit 23 stores into the input data memory 236 the Doppler image data for each sound ray and for each pixel inputted from the Doppler processing unit 22. This results in the formation of a sound ray data space regarding each set of Doppler image data within the input data memory 236.

The CPU 231 scans and converts the B-mode image data in the input data memory 236 and each set of Doppler image data with the DSC 237, and writes the converted data into the image memory 238. In this process, the Doppler image data are written as flow velocity distribution data combining the flow velocity V and the variance T, power Doppler image data using the power PW, power-plus variance Doppler image data combining the power PW and the variance T or variance image data using the variance T.

The CPU 231 writes the B-mode image data and each set of Doppler image data into different areas of the image memory 238. Then, images based on these B-mode image data and each set of Doppler image data are displayed on the image display device 4 and the display unit 7.

The B-mode image represents a tomogram of the internal tissue on the sound ray-scanned plane. Out of color Doppler images, the flow velocity distribution image serves as an image representing the two-dimensional distribution of flow velocities of the echo source. In this image, the display color is varied according to the flow direction, the brightness of the display color is varied according to the flow velocity, and the mixed quantity of a prescribed color is increased according to the variance thereby to vary the purity of the display color.

Out of color Doppler images, the power Doppler image serves as an image representing the two-dimensional distribution of the powers of Doppler signals. This image indicates the location of the echo source in motion. The brightness of the display color matches the power. Where variance is combined with it, the purity of the display color is varied by varying the mixed quantity of a prescribed color according to the variance. The variance image serves as an image representing the two-dimensional image of variance values. This image also indicates the location of the echo source in motion. The brightness of the display color matches the relative degree of variance.

When any of the images referred to above is to be displayed on the image display device 4 and the display unit 7, it is synthesized with a B-mode image in the display memory 239. The image resulting from the synthesis of the Doppler image and the B-mode image is outputted to the synthetic processing unit 25, and synthesized by this synthetic processing unit 25 with the display data of the cursors C1 and C2 and the line segment L. The data of the ultrasonic image synthesized by the synthetic processing unit 25 with the display data of the cursors C1 and C2 and the line segment L are outputted to the image display device 4 to be displayed as an image, and also displayed on the display unit 7 of the operating device 2 to enable a color Doppler image whose positional relationship with the internal tissue is clear to be observed.

Incidentally, the image to be displayed on the image display device 4 and the display unit 7 may be either a still picture or a moving picture.

Hereupon, in order to display on the display unit 7 the ultrasonic image synthesized with the display data of the cursors C1 and C2 and the line segment L by the synthetic processing unit 25, the data outputted from the synthetic processing unit 25 are transmitted from the processing device side radio communication unit 11 toward the operating device 2 via the control unit 26. In the operating device 2, the operating device side radio communication unit 8 receives the data from the processing device side radio communication unit 11, and temporarily stores them into the buffer memory 27. Then, an ultrasonic image based on the data stored in this buffer memory 27 is displayed on the display unit 7. When a moving picture is to be displayed on the display unit 7, the moving picture is enabled to be displayed by transmitting frame rate information from the processing device 5 toward the operating device 2.

When an ultrasonic image is to be displayed on the display unit 7, the ultrasonic image may be displayed either together with operating buttons (not shown), or only the ultrasonic image may be displayed without displaying operating buttons.

When measurement using the cursors C1 and C2 is to be performed on the ultrasonic image, the operator moves the cursors C1 and C2 by manipulating the pointing device 282 while watching the ultrasonic image on the display unit 7 and focuses on the tumor X which is the object of measurement. Positional information on the cursors C1 and C2 is transmitted from the operating device side radio communication unit 8 to the processing device side radio communication unit 11, and inputted to the measurement information display processing unit 24 via the control unit 26. When the positional information on the cursors C1 and C2 is inputted to the measurement information display processing unit 24, the cursor setting unit 241 generates display data of the cursors C1 and C2 and the line segment L to be displayed in a position matching the inputted positional information, and outputs them to the synthetic processing unit 25. Then, the synthetic processing unit 25 synthesizes the cursors C1 and C2 and the line segment L with the ultrasonic image, and the resultant synthetic image is transmitted from the processing device side radio communication unit 11 to the operating device side radio communication unit 8 via the control unit 26 and, when it is received by this operating device side radio communication unit 8, it is temporarily stored into the buffer memory 27 and displayed on the display unit 7.

As the cursors C1 and C2 are aligned with the object of measurement and a positioning button of the keyboard 281 is pressed, the length of the line segment L between these cursors C1 and C2 is computed by the computing unit 242 as the real length of the object of measurement on the basis of the current positional information on the cursors C1 and C2.

According to the ultrasonic diagnostic apparatus 1 described above, as the ultrasonic image can be displayed on the operating device 2, even if the operating device 2 and the image display device 4 are separate, the operator can operate the operating device 2 while watching the ultrasonic image displayed on the operating device 2, and accordingly the operator can work in a natural bodily posture.

Incidentally, it is also conceivable to equip the operating device 2 with a loudspeaker (not shown), transmit Doppler sound data taken out of echo signals by the Doppler processing unit 22 from the processing device side radio communication unit 11 to the operating device side radio communication unit 8, and reproduce the Doppler sound data received by this operating device side radio communication unit from the loudspeaker as a Doppler sound.

Further, it is also conceivable to store in advance in a memory unit (not shown) of the processing device 5 the region to be diagnosed, for instance in the operator's voice, transmit the voice data from the processing device side radio communication unit 11 to the operating device side radio communication unit 8, and aurally reproduce the information from the loudspeaker of the operating device 2.

Whereas the invention has been hitherto described with reference to the mode for implementation thereof, obviously the invention can be carried out in various modified modes without altering the essentials thereof. For instance, though not illustrate, the image display device 4 may as well have the image processing unit 23, the measurement information display processing unit 24 and the synthetic processing unit 25. In this case, the image display device 4 is provided with a radio communication unit in place of the processing device side radio communication unit 11.

Many widely different embodiments of the invention may be configured without departing from the spirit and the scope of the present invention. It should be understood that the present invention is not limited to the specific embodiments described in the specification, except as defined in the appended claims.

1. An ultrasonic diagnostic apparatus comprising:
an operating unit configured to receive instructions from an operator;
a signal processing unit configured to produce ultrasonic image data based on echo signals obtained by transmitting an ultrasonic wave; and
an image display unit configured to display an ultrasonic image based on the ultrasonic image data produced by said signal processing unit,
wherein:
said operating unit is configured as a device separate at least from said image display unit; and
said operating unit is configured to display the ultrasonic image.

2. The ultrasonic diagnostic apparatus according to claim 1, further comprising:
a data transmitting unit configured to transmit the ultrasonic image data produced by said signal processing unit to said operating unit, wherein said operating unit comprises:
a data receiving unit configured to receive the ultrasonic image data from said data transmitting unit; and
a display unit configured to display the ultrasonic image based on the ultrasonic image data received by said data receiving unit.

3. The ultrasonic diagnostic apparatus according to claim 2, wherein said display unit is configured to display buttons for enabling the operator to input the instructions.

4. The ultrasonic diagnostic apparatus according to claim 2, wherein said data transmitting unit and said data receiving unit are each configured to perform radio communication.

5. The ultrasonic diagnostic apparatus according to claim 3, wherein said data transmitting unit and said data receiving unit are each configured to perform radio communication.

6. The ultrasonic diagnostic apparatus according to claim 2, wherein said operating unit comprises a buffer memory configured to store the ultrasonic image data received by said data receiving unit, such that the ultrasonic image based on the ultrasonic image data stored in said buffer memory is displayed by said display unit.

7. The ultrasonic diagnostic apparatus according to claim 2, further comprising:
a display setting unit configured to display on the ultrasonic image a measurement range designating display which designates a measurement range of an object of measurement in the ultrasonic image; and
a computing unit configured to compute measured values based on said measurement range designating display, wherein said operating unit further comprises input means configured to move said measurement range designating display and to designate the measurement range.

8. The ultrasonic diagnostic apparatus according to claim 3, further comprising:
a display setting unit configured to display on the ultrasonic image a measurement range designating display which designates the a measurement range of an object of measurement in the ultrasonic image; and
a computing unit configured to compute measured values based on said measurement range designating display, wherein said operating, unit further comprises input means configured to move said measurement range designating display and to designate the measurement range.

9. The ultrasonic diagnostic apparatus according to claim 2, further comprising:
a processing device comprising said signal processing unit and said data transmitting unit, wherein said processing device is a separate body from said operating unit.

10. The ultrasonic diagnostic apparatus according to claim 3, further comprising:
a processing device comprising said signal processing unit and said data transmitting unit, wherein said processing device is a separate body from said operating unit.

11. The ultrasonic diagnostic apparatus according to claim 4, further comprising:
a processing device comprising said signal processing unit and said data transmitting unit, wherein said processing device is a separate body from said operating unit.

12. The ultrasonic diagnostic apparatus according to claim 5, further comprising:
a processing device comprising said signal processing unit and said data transmitting unit, wherein said processing device is a separate body from said operating unit.

13. The ultrasonic diagnostic apparatus according to claim 6, further comprising:
a processing device comprising said signal processing unit and said data transmitting unit, wherein said processing device is a separate body from said operating unit.

14. The ultrasonic diagnostic apparatus according to claim 7, further comprising:
a processing device comprising said signal processing unit and said data transmitting unit, wherein said processing device is a separate body from said operating unit.

15. The ultrasonic diagnostic apparatus according to claim 7, further comprising:
a processing device comprising said signal processing unit, said data transmitting unit, said display setting unit, and said computing unit, wherein said processing device is a separate body from said operating unit.

16. The ultrasonic diagnostic apparatus according to claim 1, wherein:
said signal processing unit is configured to produce Doppler sound data by taking out Doppler components from the echo signals and to transmit the Doppler sound data to said operating unit, said operating unit comprising a loudspeaker configured to reproduce Doppler sounds based on the Doppler sound data.

17. The ultrasonic diagnostic apparatus according to claim 1, wherein the ultrasonic image displayed on said operating device is a moving image.

18. An operating device of an ultrasonic diagnostic apparatus, said operating device configured to:
receive instructions from an operator said operating device communicatively coupled to a signal processing unit configured to produce ultrasonic image data based on echo signals obtained by transmitting an ultrasonic wave, and an image display unit configured to display an ultrasonic image based on the ultrasonic image data, said operating device being a body positioned separate from at least the image display unit said operating device wherein it is enabled also configured to display the ultrasonic image.

19. The operating device of an ultrasonic diagnostic apparatus according to claim 18, further comprising:
a data receiving unit configured to receive the ultrasonic image data produced by the signal processing unit; and
a display unit configured to display the ultrasonic image based on the ultrasonic image data received by said data receiving unit.

20. The operating device of an ultrasonic diagnostic apparatus according to claim 19, further comprising:
a buffer memory configured to store the ultrasonic image data received by said data receiving unit, wherein the ultrasonic image based on the ultrasonic image data stored in said buffer memory is displayed on said display unit.
